SEQUENCE LISTING (9 sequences)

SEQ ID NO: 1 - DNA, 18 bp, Artificial Sequence
  Synthetic Sequence
ccaatgcgcgacattccg

SEQ ID NO: 2 - DNA, 18 bp, Artificial Sequence
  Synthetic Sequence
acttccatgccaatgcgc

SEQ ID NO: 3 - DNA, 18 bp, Artificial Sequence
  Synthetic Sequence
ctcatggattgttgcaat

SEQ ID NO: 4 - DNA, 18 bp, Artificial Sequence
  Synthetic Sequence
ctgcggtaaggcttgagt

SEQ ID NO: 5 - DNA, 18 bp, Artificial Sequence
  Synthetic Sequence
aatttgatggcacctgtg

SEQ ID NO: 6 - DNA, 18 bp, Artificial Sequence
  Synthetic Sequence
tgagttgagtcagcactg

SEQ ID NO: 7 - DNA, 18 bp, Artificial Sequence
  Synthetic Sequence
ttgtgttacattgtatgc

SEQ ID NO: 8 - DNA, 18 bp, Artificial Sequence
  Synthetic Sequence
gcgcgacattccgaagaa

SEQ ID NO: 9 - DNA, 21 bp, Artificial Sequence
  Synthetic Sequence
tgtagcacgattgcagcattg

(19) United States
(12) Patent Application Publication
(10) Pub. No.: US 2023/0416849 A1
(43) Pub. Date: Dec. 28, 2023

(54) METHODS FOR DETECTING VIRAL RNA USING ATP-RELEASING NUCLEOTIDES

(71) Applicant: The Board of Trustees of the Leland Stanford Junior University, stanford, CA (US)

(72) Inventors: Eric T. Kool, Stanford, CA (US); Michael G. Mohsen, Stanford, CA (US)

(21) Appl. No.: 18/037,454

(22) PCT Filed: Nov. 23, 2021
(86) PCT No.: PCT/US2021/060535

§ 371 Date: May 17, 2023

Related U.S. Application Data
(60) Provisional application No. 63/117,640, filed on Nov. 24, 2020.

(51) Int. Cl. C12Q 1/70

(52) U.S. Cl. CPC C12Q 1/701

(57) ABSTRACT

Compositions and methods are provided for a biochemical method for detecting virus RNA sequences, either after isolation or directly in patient samples, by detecting ATP released from an ATP-releasing nucleotide (ARN) during synthesis. In some embodiments the virus is a coronavirus, e.g. SARS-CoV2.

### CROSS-REFERENCE TO RELATED APPLICATIONS

This application claims the benefit of U.S. provisional Patent Application Ser. No. 63/117,640, filed on Nov. 24, 2020, the contents of which are herein incorporated by reference in their entirety.

### INCORPORATION BY REFERENCE OF SEQUENCE LISTING PROVIDED AS A TEXT FILE

A Sequence Listing is provided herewith in a text file, (STAN-1773WO_SeqListing_ST25), created on Nov. 22, 2021, and having a size of 2000 bytes. The contents of the text file are incorporated herein by reference in its entirety.

### STATEMENT REGARDING FEDERALLY SPONSORED RESEARCH

This invention was made with Government support under contract CA217809 awarded by the National Institutes of Health. The Government has certain rights in the invention.

### BACKGROUND

Culture-based systems for virus isolation have been the standard in clinical virology for many years. However, the isolation of viruses in culture is slow, time-consuming, and labor-intensive and lacks the sensitivity needed to have an appreciable impact on clinical decision making. Many clinically relevant viruses are simply difficult to grow or cannot be grown at all in cultured cells, while other viruses require specialized culture systems that are either not available or too complicated for routine use in diagnostic laboratories.

The accuracy and timeliness of viral diagnosis have been improved with the development and implementation of molecular amplification methods, such as PCR, particularly real-time PCR, and other molecular amplification technologies such as nucleic acid sequence-based amplification, strand displacement amplification, transcription-mediated amplification, branched DNA amplification, loop-mediated amplification, and helicase-dependent amplification. Molecular amplification methods are especially well suited for detecting viruses present in small specimen volumes or that are in low numbers within clinical specimens.

There are significant downsides, however, to reverse-transcription polymerase chain reaction for the detection of virus in large numbers of samples, as is required for COVID-19 testing. The assays require trained personnel, specific chemical supplies and expensive instruments that take hours to provide results and are often available only in labs that provide routine, centralized services. This limits the number of tests that can be done, especially in developing countries. Scaling up reliable tests quickly has proved challenging, too: some early RT-PCR tests malfunctioned, for example, leading to a series of delays.

Rapid and simple detection of viral RNAs, including SARS-CoV2, is important to human health. Current methods are too slow, expensive, specialized, and inaccurate. The present disclosure addresses this issue. Publications of interest include US-2017/0159112A1, herein specifically incorporated by reference.

### SUMMARY

Compositions and methods are provided for a biochemical method for detecting viral RNA sequences, either after isolation or directly in patient samples, by use of luminescence. A sample suspected of comprising viral RNA is introduced into a reaction mixture comprising with at least one chimeric nucleoside tetraphosphate dimer in which ATP is the leaving group, referred to as ATP-releasing nucleotides (ARNs), virus-specific primer or primers, and a reverse transcriptase (RT) enzyme. Replication of the viral RNA by the reverse transcriptase incorporates ARN into the transcribed polynucleotide, thereby releasing ATP.

The released ATP can be assayed in a qualitative or quantitative analysis, where one equivalent of ATP is released for every deoxynucleotide incorporated from an ARN. Any convenient method for the detection of ATP can be used, as known in the art, including without limitation: luciferase bioluminescence assays, fluorescent dyes, target-responsive aptasensors, and the like. In some such embodiments, the detection reagent(s) is combined with the reaction mixture after the polymerization reaction is substantially complete, e.g. where a desired level of the product of the reaction has accumulated, such as after at least about 10 minutes, at least about 15 minutes, after at least about 30 minutes or more. In some embodiments a reaction time is of about 10 minutes. In some embodiments, released ATP is detected by detecting light produced by luciferase in the presence of ATP and luciferin.

A reaction mixture comprises a combination of dNTPs and ARNs that is sufficient to provide a substrate for all bases present in the target RNA virus sequence. Generally all four deoxynucleotides are present in a reaction mix, where each deoxynucleotide is provided either as a native dNTP, or as an ARN, e.g. deoxyadenosine-5′-tetraphosphate-P4-5′-adenosine (dCppppA), deoxycytidine-5′-tetraphosphate-P4-5′-adenosine (dAppppA), deoxyguanosine-5′-tetraphosphate-P4-5′-adenosine (dGppppA) or deoxythymidine-5′-tetraphosphate-P4-5′-adenosine (dTppppA). In some embodiments a reaction mixture comprises ATP-depleted dCp₄ A, which reduces background. In some embodiments a reaction mixture comprises as the sole nucleotides ATP-depleted dCp₄A,dTp₄A, dATP, and dGTP.

The reaction mixture comprises an effective concentration of an RT enzyme with the ability to efficiently accept ARNs as substrates. In some embodiments the RT is an ultra-processive reverse transcriptase, e.g. a group II intron-encoded RT, an engineered MMLV mutant RT. etc. The RT in some embodiments is SuperScript IV™ (SSIV). In some such embodiments an extension temperature of about 55° C. is used.

Primers are designed to be complementary to the virus of interest. In some embodiments the sample suspected of comprising virus RNA and the primers are heated to about 85° C. for about 5 minutes and flash-cooled, e.g. on ice or cold block. In some embodiments the concentration of primers is from about 0.5 μM to about 10 μM, e.g. about 1 μM, about 2 μM about 5 μM. In some embodiments a plurality of primers are included in the reaction mixture, e.g. at least 2, 3, 4, 5, 6, 7, 8, 9 or more different primers, each complementary to the virus sequence of interest. In some embodiments the RNA virus is SARS-CoV2, where the primers include, without limitation, those provided in Table 1.

Applications for methods of the invention include in vitro diagnostics, including clinical diagnostics, research in the fields of molecular biology, high throughput drug screening, veterinary diagnostics, environmental testing, etc. In vitro diagnostics and clinical diagnostics relate to the analysis of nucleic acid samples drawn from the body to detect the existence of a disease or condition, its stage of development and/or severity, and the patient's response to treatment.

### BRIEF DESCRIPTION OF THE DRAWINGS

FIG. 1. Comparing signal generated from two different ultraprocessive RTs: SuperScript IV (SSIV) and MarathonRT. In the presence of 50 nM SARS-CoV-2 RNA fragments and 100 nM N2 primer, SSIV produced approximately 6-fold greater luminescence signal relative to Marathon RT.

FIG. 2. Effect of extension time on specificity. As incubation time at 50° C., specificity for SARS-CoV-2 vs. SARS-CoV RNA sequences decreased from 4.2-fold (10 min) to 2.0-fold (60 min). 50 nM SARS-CoV-2/SARS-CoV RNA sequences and 100 nM N2 primer were tested.

FIG. 3. Increased specificity and sensitivity incubating with incubation at 55° C. relative to 50° C. 50 nM SARS-CoV-2/SARS-CoV RNA sequences and 100 nM N2 primer were tested.

FIG. 4. Using an annealing step (incubated at 65° C. for 5 minutes, then flash cooled on ice), increased concentration of virus-specific primer 6 (VSP-6) from 0.1 μM to 10 μM resulted in increased sensitivity.

FIG. 5. Testing different temperatures for annealing step. Tubes containing 50 nM RNA targets and 1 μM VSP-6 primer were incubated at the indicated temperature for 5 minutes prior flash cooling on ice and 10-minute extension with RT. Annealing at 85° C. resulted in increased sensitivity.

FIG. 6. Testing different incubation times for the annealing step. Tubes containing 50 nM RNA targets and 1 μM VSP-6 primer were incubated at 85° C. Increasing the incubation time resulted in decreased signals, presumably because of decomposition of RNA targets.

FIG. 7. Using a combination of specific primers to increase sensitivity. A 9-primer cocktail (9PC, 1 μM each) provided a 2.5-fold increase in sensitivity relative to N2 (1 μM). The 9-primer cocktail contained VSPs 1-7, N2, and N3.

FIG. 8. Optimizing nucleotide combinations. dTp₄A/dCp₄A/dATP/dGTP (20 μM each) provided a signal:noise ratio of 25:1, almost double that of dAp₄A/dGp₄A/dTTP/dCTP (20 μM each), which provided a signal:noise ratio of 13:1. 50 nM of target RNA was detected using a 9-primer cocktail (1 μM each; VSPs 1-7, N2, N3).

FIG. 9. Spiking in 50 nM SARS-CoV-2 RNA fragment into 1 μg of total RNA isolated from human lung tissue. First plus or minus refers to addition of RNA, second plus or minus refers to addition of 9-primer cocktail (1 μM each; VSPs 1-7, N2, N3). This result shows that non-specific priming was not an issue, even in the presence of 1 μg of total RNA.

FIG. 10. Specificity for SARS-COV-2 vs. related coronavirus sequences (SARS-CoV, MERS-CoV) with 50 nM RNA targets, a 9-primer cocktail (1 μM each; VSPs 1-7, N2, N3) and 1 μg of total RNA isolated from human lung tissue. Specificity for SARS-CoV-2 vs. SARS-CoV was 1.8-fold; however, we hypothesize that this could be significantly improved by optimizing the combination of primers. The specificity for SARS-CoV-2 vs. MERS-CoV was 22-fold.

### DETAILED DESCRIPTION

Before embodiments of the present disclosure are further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Any methods and materials similar or equivalent to those described herein can also be used in the practice or testing of embodiments of the present disclosure.

It must be noted that as used herein and in the appended claims, the singular forms “a”, “and”, and “the” include plural referents unless the context clearly dictates otherwise. Thus, for example, reference to “a compound” includes not only a single compound but also a combination of two or more compounds, reference to “a substituent” includes a single substituent as well as two or more substituents, and the like.

In describing and claiming the present invention, certain terminology will be used in accordance with the definitions set out below. It will be appreciated that the definitions provided herein are not intended to be mutually exclusive. Accordingly, some chemical moieties may fall within the definition of more than one term.

As used herein, the phrases “for example,” “for instance,” “such as,” or “including” are meant to introduce examples that further clarify more general subject matter. These examples are provided only as an aid for understanding the disclosure, and are not meant to be limiting in any fashion.

As used herein, the terms “determining,” “measuring,” “assessing,” and “assaying” are used interchangeably and include both quantitative and qualitative determinations.

The terms “nucleic acid molecule” and “polynucleotide” are used interchangeably and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

As used herein, “suitable conditions” for carrying out a synthetic step are explicitly provided herein or may be discerned by reference to publications directed to methods used in synthetic organic chemistry. The reference books and treatise set forth above that detail the synthesis of reactants useful in the preparation of compounds of the present invention, will also provide suitable conditions for carrying out a synthetic step according to the present invention.

As used herein, “methods known to one of ordinary skill in the art” may be identified through various reference books and databases. Suitable reference books and treatise that detail the synthesis of reactants useful in the preparation of compounds of the present invention, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Specific and analogous reactants may also be identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs may be prepared by custom chemical synthesis houses, where many of the standard chemical supply houses (e.g., those listed above) provide custom synthesis services.

The compositions and methods of the present invention provide for improved detection and diagnosis of pathogenic viruses, particularly viruses that infect avians and mammals for medical and veterinary use. Viruses include those that infect, e.g. farm animals including horses, cattle, sheep, pigs, chickens, turkeys, etc., domestic animals including dogs and cats; and viruses that infect humans.

In some embodiments a virus detected by the methods of the invention is an RNA virus. An RNA virus is a virus that has RNA (ribonucleic acid) as its genetic material. This nucleic acid is usually single-stranded RNA (ssRNA) but may be double-stranded RNA (dsRNA). Human diseases caused by RNA viruses include without limitation AIDS, Ebola hemorrhagic fever, SARS, COVID19, influenza, hepatitis C, West Nile fever, polio, and measles.

The ICTV classifies RNA viruses as those that belong to Group III, Group IV or Group V of the Baltimore classification system of classifying viruses and does not consider viruses with DNA intermediates in their life cycle as RNA viruses. Viruses with RNA as their genetic material but that include DNA intermediates in their replication cycle are retroviruses, and comprise Group VI of the Baltimore classification. Notable human retroviruses include HIV-1 and HIV-2, the cause of the disease AIDS. For the purposes of the present invention, an RNA virus is one that is within Group III, IV, V or VI unless otherwise indicated.

The double-stranded (ds)RNA viruses represent a diverse group of viruses that vary widely in host range, genome segment number, and virion organization. Members of this group include the rotaviruses and picobirnaviruses. The clades include the Caliciviridae, Flaviviridae, and Picornaviridae families, and a second that includes the Alphatetraviridae, Birnaviridae and Cystoviridae, Nodaviridae, and Permutotretraviridae families. Double-stranded RNA viruses (Group III) contain from one to a dozen different RNA molecules, each coding for one or more viral proteins.

RNA viruses can be further classified according to the sense or polarity of their RNA into negative-sense and positive-sense, or ambisense RNA viruses. Positive-sense ssRNA viruses (Group IV) have their genome directly utilized as if it were mRNA, with host ribosomes translating it into a single protein that is modified by host and viral proteins to form the various proteins needed for replication. One of these includes RNA-dependent RNA polymerase (RNA replicase), which copies the viral RNA to form a double-stranded replicative form. In turn this directs the formation of new virions. Viruses in this group include I. Bymoviruses, comoviruses, nepoviruses, nodaviruses, picornaviruses, potyviruses, sobemoviruses and a subset of luteoviruses (beet western yellows virus and potato leafroll virus)—the picorna like group (Picornavirata); II. Carmoviruses, dianthoviruses, flaviviruses, pestiviruses, tombusviruses, hepatitis C virus and a subset of luteoviruses (barley yellow dwarf virus)—the flavi like group (Flavivirata); III. Alphaviruses, carlaviruses, furoviruses, hordeiviruses, potexviruses, rubiviruses, tobraviruses, tricornaviruses, tymoviruses and hepatitis E virus—the alpha like group (Rubivirata). Alphaviruses and flaviviruses can be separated into two families—the Togaviridae and Flaviridae.

Coronavirus are of particular interest, e.g. SARS-CoV1, SARS-CoV2; MERS-CoV, etc. SARS-CoV2 primer sequences may be selected to complement one or more circulating variants of the virus.

Negative-sense ssRNA viruses (Group V) must have their genome copied by an RNA replicase to form positive-sense RNA. The positive-sense RNA molecule then acts as viral mRNA, which is translated into proteins by the host ribosomes. The resultant protein goes on to direct the synthesis of new virions, such as capsid proteins and RNA replicase, which is used to produce new negative-sense RNA molecules. Group V-negative-sense ssRNA viruses include one order and eight families in this group. The group includes a number of clinically relevant pathogens. Bornaviridae-Borna disease virus; Family Filoviridae—includes Ebola virus, Marburg virus; Family Paramyxoviridae—includes Measles virus, Mumps virus, Nipah virus, Hendra virus, RSV and NDV; Family Rhabdoviridae—includes Rabies virus; Family Nyamiviridae—includes Nyavirus; Family Arenaviridae—includes Lassa virus; Family Bunyaviridae—includes Hantavirus, Crimean-Congo hemorrhagic fever; Family Ophioviridae; Family Orthomyxoviridae—includes Influenza viruses; Genus Deltavirus—includes Hepatitis D virus; Genus Dichorhavirus; Genus Emaravirus; Genus Nyavirus—includes Nyamanini and Midway viruses; Genus Tenuivirus; Genus Varicosavirus

Retroviruses (Group VI) have a single-stranded RNA genome although they use DNA intermediates to replicate. Reverse transcriptase, a viral enzyme that comes from the virus itself after it is uncoated, converts the viral RNA into a complementary strand of DNA, which is copied to produce a double-stranded molecule of viral DNA. After this DNA is integrated into the host genome using the viral enzyme integrase, expression of the encoded genes may lead to the formation of new virions. Included in retroviruses are the lentiviruses, e.g. HIV-1 and HIV-2.

ATP-releasing nucleotides (ARNs). As used herein, the term ARN refers to a chimeric DNA nucleoside tetraphosphate dimer comprising ATP. Use of one or more ARN as a substrate for a template dependent polymerization reaction results in the incorporation of the dNMP substituent into the elongating primer or template, and the corresponding release of the ATP substituent. As shown with reference to formula (I), the ARN compounds contain an adenosine substituent linked via four phosphate groups to a 2′-deoxynucleoside substituent. ARNs have the general structure:

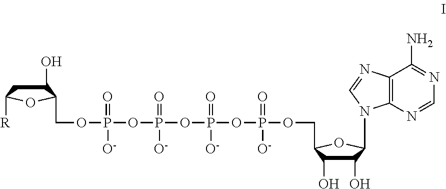

where R is any purine or pyrimidine including substituted purines or pyrimidines. R groups of interest include adenine (A), thymine (T), guanine (G), cytosine (C), or an analog thereof, where an analog has a modified base retains an ability to base pair with a complementary nucleotide. These ARNs may also be referred to individually as, for example, deoxyadenosine-5′-tetraphosphate-P4-5′-adenosine (dCppppA), deoxycytidine-5′-tetraphosphate-P4-5′-adenosine (dAppppA), deoxyguanosine-5′-tetraphosphate-P4-5′-adenosine (dGppppA) or deoxythymidine-5′-tetraphosphate-P4′-tetraphosphate-P4-5′-adenosine (dTppppA). While drawn as phosphate anions, it is understood that they may be protonated at lower pH values.

In some embodiments a reaction mix comprises an ARN that is ATP depleted, i.e. contaminating free ATP has been depleted from the ARN. Various methods known in the art for depletion of ATP may be used for this purpose, including, without limitation, purification with HPLC to separate ATP from the ARN; reaction of the ARN with luciferase and luciferin to selectively consume residual ATP without affecting dCp4A; and the like. In some embodiments the ATP depleted ARN is dCp₄A.

The terms “nucleoside”, “nucleotide”, “deoxynucleoside”, and “deoxynucleotide” are intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the “nucleoside”, “nucleotide”, “deoxynucleoside”, and “deoxynucleotide” include those moieties that contain not only conventional ribose and deoxyribose sugars, but other sugars as well.

Nucleotides useful in the invention include naturally occurring, or native, nucleotides and nucleotide analogs. Exemplary nucleotides include phosphate esters of deoxyadenosine, deoxycytidine, deoxyguanosine, deoxythymidine, deoxyuridine, adenosine, cytidine, guanosine, and uridine. Other nucleotides comprise an adenine, cytosine, guanine, thymine base, a xanthine or hypoxanthine; 5-bromouracil, 2-aminopurine, deoxyinosine, or methylated cytosine, such as 5-methylcytonsine, and N4-methoxydeoxycytosine. Deoxynucleotide analogues useful in the invention include, without limitation, -5 alkyl, alkenyl, alkynyl, and F, CI, Br, I pyrimidines, and the same substituents at C7 of 7-deazapurines; 5-methyl C, 5-hydroxymethyl C.

The term a “native dNTP” refers to naturally occurring deoxyribose nucleotide triphosphosphates, as known in the art, e.g. dTTP, dATP, dCTP, dGTP.

“Modified nucleotides”, “modified nucleosides”, “nucleotide analogs”, or “nucleoside analogs” (excluding A, T, G, and C) include for example, nucleotides or nucleosides having a structure derived from purine or pyrimidine (i.e., nucleotide or nucleoside analogs). For example and without limitation, a modified adenine may have a structure including a purine with a nitrogen atom covalently bonded to C6 of the purine ring as numbered by conventional nomenclature known in the art. In addition, it is recognized that modifications to the purine ring and/or the C6 nitrogen may also be included in a modified adenine. A modified thymine may have a structure comprising at least a pyrimidine, an oxygen atom covalently bonded to the C4 carbon, and a C5 methyl group. Again, it is recognized by those skilled in the art that modifications to the pyrimidine ring, the C4 oxygen and/or the C5 methyl group may also be included in a modified adenine. For example and without limitation, a modified guanine may have a structure comprising at least a purine, and an oxygen atom covalently bonded to the C6 carbon. A modified cytosine may have a structure including a pyrimidine and a nitrogen atom covalently bonded to the C4 carbon. Modifications to the purine ring and/or the C6 oxygen atom may also be included in modified guanine nucleotides or nucleosides. Other known modifications to purines include 7-deaza derivatives, such as 7-deazaadenine and 7-deazaguanine. Modifications to the pyrimidine ring and/or the C4 nitrogen atom may also be included in modified cytosine nucleotides or nucleosides.

The ARN compounds may be made by the methods disclosed in, for example, US-2017-0159112-A1, herein specifically incorporated by reference, for example where salts of deoxynucleoside monophosphates (dNMPs) are activated and then reacted with a salt of 5′-ATP. Alternatively the ARN compounds are made by a method where a salt of adenosine monophosphate (AMP) is activated and then reacted with the salts of different deoxynucleotide-5′-triphosphates (dNTPs).

A “target sequence” or “sequence of interest” refers to the particular nucleotide sequence of the RNA virus of interest that can be hybridized to a primer or complementary template. While the target polynucleotide may be single stranded or double-stranded in its native state, typically it will be denatured prior to contacting with a primer or template.

The term “nucleic acid” and “polynucleotide” are used interchangeably herein to describe a polymer of any length, e.g., greater than about 10 bases, greater than about 100 bases, greater than about 500 bases, greater than 1000 bases, usually up to about 10,000 or more bases composed of nucleotides, e.g., ribonucleotides.

As used herein, a “test sample” is a sample suspected of comprising viral RNA to be analyzed for the presence or amount of the target sequence. The test sample may be of any biological origin, including any tissue or polynucleotide-containing material obtained from a human, such as one or more of nasopharyngeal swabs, nasal swabs, saliva, tissue or organ lavage, sputum, peripheral blood, plasma, serum, bone marrow, biopsy tissue including lymph nodes, respiratory tissue or exudates, gastrointestinal tissue, cervical swab samples, semen or other body fluids, tissues or materials. Biological samples may be treated to disrupt tissue or cell structure, thereby releasing intracellular components into a solution which may contain enzymes, buffers, salts, detergents and the like. Alternative sources of nucleic acids may include water or food samples that are to be tested for the presence of a particular analyte polynucleotide that would indicate the presence of an RNA virus. A test sample may comprise DNA, RNA, etc., including total mixed RNA from a biological sample, purified RNA subsets such as mRNA, rRNA etc.

The term “primer” means an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process are determined by the sequence of the template polynucleotide. A primer serves as an initiation point for nucleotide polymerization catalyzed by reverse transcriptase. In the methods of the invention, a primer is usually complementary to a target sequence.

Primers are usually of a sufficient length to specifically hybridize to, and initiate synthesis from, the target polynucleotide. A primer can be, for example, of at least about 6 bases in length, more usually at least 7, 8, or 9 bases; for many embodiments of the invention, oligonucleotides are at least 10 bases, at least 12 bases, at least about 14 bases, at least about 16 bases, and not more than about 50 bases in length, usually not more than about 30 bases in length, not more than 25 bases in length, or any length range between any two of these lengths.

As is known in the art, a primer may further comprise a non-complementary region, e.g. to provide for indexing, bar-coding, tags, and the like.

Primers may comprise native nucleic acids, e.g. DNA or RNA, or may comprise modified nucleotides, for example to enhance stability of hybridization. Modified nucleic acids of interest include, without limitation, locked nucleic acid (LNA), 2′-O-methyl RNA, etc.

With respect to the region of complementarity between a primer or template and a target, the sequence may or may not be completely complementary. If not completely complementary, the target and primer or template are at least substantially complementary, such that the number of mismatches allows specific priming of DNA synthesis. The region of complementarity is usually at least about 6 bases in length, more usually at least 7, 8, or 9 bases; for many embodiments of the invention, at least 10 bases, at least 12 bases, at least about 14 bases, at least about 16 bases, and not more than about 50 bases in length, usually not more than about bases in length, not more than 25 bases in length, or any length range between any two of these lengths. Over the region of complementarity the number of mismatches will usually not be more than about 15% of the total number, not more than about 10%, of the total, not more than about 5% of the total. In other words, the region of complementarity will be at least about 85% identical to the target sequence, at least about 90% identical, at least about 95% identical, and may be 100% identical.

The sequence of a primer or template is selected to be complementary, competitive, mismatched, etc. with respect to a target sequence, as dictated by the specific interests of the method. A highly selective probe binds with high preference to the exact complementary sequence on a target strand as compared to a sequence that has one or more mismatched bases. Less selective probes are also of interest for some embodiments, where hybridization is sufficient for detectable reactions to occur in the presence of one, two three or more mismatches, where a mismatch may include substitutions, deletions, additions, etc.

The phrase “primer extension conditions” denotes conditions that permit for RT mediated primer extension by addition of nucleotides to the end of the primer molecule using the template strand as a template.

The term “complementary, “complement,” or “complementary nucleic acid sequence” refers to the nucleic acid strand that is related to the base sequence in another nucleic acid strand by the Watson-Crick base-pairing rules. In general, two sequences are complementary when the sequence of one can hybridize to the sequence of the other in an anti-parallel sense wherein the 3′-end of each sequence hybridizes to the 5′-end of the other sequence and each A, T, G, and C of one sequence is then aligned with a T, A, C, and G, respectively, of the other sequence.

The term “duplex” means at least two oligonucleotides and/or polynucleotides that are fully or partially complementary undergo Watson-Crick type base pairing among all or most of their nucleotides so that a stable complex is formed. The terms “annealing” and “hybridization” are used interchangeably to mean the formation of a stable duplex. “Perfectly matched” in reference to a duplex means that the poly- or oligonucleotide strands making up the duplex form a double stranded structure with one another such that every nucleotide in each strand undergoes Watson-Crick base pairing with a nucleotide in the other strand. The term “duplex” may include the pairing of nucleoside analogs, such as deoxyinosine, nucleosides with 2-aminopurine bases, and the like, that may be employed. A “mismatch” in a duplex between two oligonucleotides or polynucleotides means that a pair of nucleotides in the duplex fails to undergo Watson-Crick bonding.

The terms “hybridization”, and “hybridizing”, in the context of nucleotide sequences are used interchangeably herein. The ability of two nucleotide sequences to hybridize with each other is based on the degree of complementarity of the two nucleotide sequences, which in turn is based on the fraction of matched complementary nucleotide pairs. The more nucleotides in a given sequence that are complementary to another sequence, the more stringent the conditions can be for hybridization and the more specific will be the hybridization of the two sequences. Increased stringency can be achieved by elevating the temperature, increasing the ratio of co-solvents, lowering the salt concentration, and the like.

ATP detection reagent(s). Many reagents and assays are known in the art for use in detecting the presence of ATP. For the purposes of the present invention, these reagents are used to detect ATP released during DNA synthesis, and thus provide a qualitative or quantitative assessment for the presence of the target polynucleotide sequence. ATP detection reagents include without limitation luciferase bioluminescence assays (see, for example. J Appl Biochem 3, 473 (1981); Fraga (2008) Photochemical & Photobiological Sciences 7(2):146-158; Bell et al. (2007) Methods Cell Biol. 80:341-352), fluorescent dyes, target-responsive aptasensors, glass bead microarray, GO-nS nanocomplex platform, and the like. In certain embodiments the assay utilizes detection of light produced by luciferin and luciferase.

Exemplary fluorescent dyes are described, for example in Jose et al. (2007) Org. Lett. 9:1979-1982; Lee et al. (2004) Angew. Chem. Int. Ed. 43:4777-4780; Sancenon et al. (2001) Angew. Chem. Int. Ed. 40:2640-2643; Mizukami et al. (2002) JACS 124:3920-3925; Schneider et al. (2000) JACS 122:542-543; Ojida et al. (2006) Angew. Chem. Int. Ed. 45:5518-5521; Li et al. (2005) Angew. Chem. Int. Ed. 44:6371-6374, each of which is herein specifically incorporated by reference.

Target responsive aptamers are described, for example, by Li & Ho (2008) JACS 130:2380-2381; Li & Lu (2006) Angew. Chem. Int. Ed. 45:90-94; Zayats (2006) JACS 128:13666-13667. Glass bead microarrays are described by McClesky et al. (2003) JACS 125:1114-1115. A GO-nS nanocomplex platform is described by Wang et al. (2013) Anal. Chem. 85:6775-6782. Each of these references is herein specifically incorporated by reference.

The term “luciferase” refers to an adenosine triphosphate (ATP) hydrolase that catalyzes the hydrolysis of ATP into constituent adenosine monophosphate (AMP) and pyrophosphate (PPi) along with the release of light. A luciferase has an activity described as EC 1.13.12.7, according to IUBMB enzyme nomenclature. A luciferase of interest is Photinus luciferin 4-monooxygenase (ATP-hydrolyzing).

Luciferin is a common bioluminescent reporter used for in vitro assays in combination with luciferase. This water soluble substrate for the Firefly luciferase enzyme (e.g. Photinus pyralis, Cypridina, Gaussia, Renilla, etc.) utilizes ATP and Mg²⁺ as co-factors to emit a characteristic yellow-green emission in the presence of oxygen. Many reagents and kits are commercially available for this purpose. When luciferin and luciferase are combined in a reaction mixture comprising ATP, there is an immediate flash of light that reaches peak intensity within 0.3-0.5 seconds. The light then begins to decay rapidly with a half-life around 0.5-1.0 min. The optional addition of Coenzyme A to the reaction mixture prevents the fast reaction decay, extending the half-life of the reaction from 2-5 minutes. Variations of luciferin are also known that yield slower signal generation for convenience.

The term “reagent mix”, as used herein, refers to a combination of reagents, that are interspersed and not in any particular order. A reagent mix is heterogeneous and not spatially separable into its different constituents. Examples of mixtures of elements include a number of different elements that are dissolved in the same aqueous solution, or a number of different elements attached to a solid support at random or in no particular order in which the different elements are not spatially distinct.

### COMPOSITIONS

In some embodiments of the invention, a reaction mixture, or certain components thereof, is provided, which mixture comprises the components required for detecting the presence of a target RNA virus sequence of interest by a polymerization reaction, where the reaction mixture includes at least one chimeric nucleoside tetraphosphate dimer in which ATP is the leaving group. In some such embodiments the RNA virus is SARS-CoV2, where the primer sequences optionally include one or more of the primer sequences set forth in Table 1.

In some embodiments, two ARNs are provided with 2 dNTPs. While any combination can be used, e.g. by optimizing with a polymerase of interest, in some embodiments a preferred combination comprises as the sole nucleotides ATP-depleted dCp₄A,dTp₄A, dATP, and dGTP. The ARN and dNTP in a reaction mixture are typically provided at a working concentration, which may be empirically determined, for example at a concentration of from about 0.1 JIM for each dNTP or ARN, at least about 1 μM, at least about 10 μM, at least about 20-25 μM, at least about 35 μM, at least about 50 μM, up to about 75 μM, up to about 100 μM, up to about 250 μM, up to about 500 μM, or more.

In some embodiments the reagent ARN and dNTPs can be provided in a concentrated form, suitable for dilution into a reaction mixture, where the ARN and dNTP reagents may be pre-mixed or separately formulated.

A reaction mixture will also comprise a polymerase at an appropriate concentration to perform the synthetic reaction, e.g. using commercially available enzymes according to the manufacturer's instructions. The Michaelis-Menten constant (Kₘ) of an ARN for the polymerase may be comparable to the Kₘ of natural dNTPs, for example less than about 20 μM. The kcat values for ARNs are within about 2-fold, within about 1.5-fold of those of native nucleotides, and may be used with enzymes where the values are within about 20-fold those of native dNTPs. Reverse transcriptase enzymes, including without limitation, ultra-processive engineered MMLV reverse transcriptase, are suitable for this purpose. In some embodiments a suitable enzyme can be provided in a kit, with the nucleotide reagents.

A reaction mixture will comprise the sample suspected of comprising the target RNA virus sequence. The polynucleotides present in the sample are usually denatured according to methods known in the art prior to contacting with the polymerase. Any sample can be analyzed, including without limitation biological samples from an individual or population, swabs of potentially contaminated surfaces, and the like. Detection of a target sequence can be accomplished with as little as 1 pM quantities, for example at about 1 pM, 5 pM, 10 pM, 100 pM, 250 pM, 500 pM, 1 nM, 5 nM, 10 nM or more.

The reaction mixture will comprise a template or primer to initiate polymerization, where the template or primer comprises a sequence complementary to the target sequence. Parameters for primer or template are as defined herein. The concentration of template or primer is determined by the specific requirements of the analysis, but is usually at least about 1 pM, at least about 0.5 nM, at least about 1 nM, and may be from about 1 nM to about 100 μM, from about 1 nM to about 10 μM, from about 1 nM to about 1 μM, using guidelines known in the art for the polymerase and similar reaction conditions.

The reaction mixture also comprises buffers, salts, etc. as known in the art and appropriate for the polymerase or reverse transcriptase. Inhibitors of nucleases, etc. can also be added. The temperature of the reaction is generally between about 20° C. and 40° C. The pH of the reaction is generally between pH 6 and pH 9. These ranges may be extended.

When changing the concentration of a particular component of the reaction medium, that of another component may be changed accordingly. For example, the concentrations of several components such as nucleotides templates or primers may be simultaneously controlled in accordance with the change in those of other components. Also, the concentration levels of components in the reactor may be varied over time.

The reactions may be multiplexed to perform a plurality of simultaneous syntheses, utilizing such reaction vessels as 96 well plates, etc., as are known in the art.

The ATP produced by the polymerase reaction can be detected in any of a variety of different ways. The released ATP can be accumulated in the reaction mixture and then detected by the addition of ATP-dependent detection reagent(s). In such embodiments the detection reagents are added to the reaction mixture after a period of time and under such conditions that the polymerization reaction has proceeded to a desired degree, e.g. to exhaustion of the substrate or primer, or to an intermediate stage pre-determined for the assay. Usually in such embodiments a reaction proceeds for at least about 10 minutes, at least about 15 minutes, at least about 30 minutes, at least about 1 hour, at least about 2 hours, at least about 4 hours, at least about 6 hours, at least about 8 hours, at least about 12 hours, at least about 18 hours, at least about 24 hours or more. In some embodiments a reaction time is of about 10 minutes. Included are, for example, a reaction of about 10 minutes to 24 hours, from about 10 minutes to about 12 hours, etc.

In other embodiments, the ATP detection reagent, e.g. a fluorescent dye, chemiluminescent system, aptamer, etc. is included in the reaction mixture at initiation, and measurement of the signal is detected during the polymerization reaction. Regardless of when the detection reagent is included, the concentration, buffer, conditions, etc. are chosen to be appropriate for the reagent.

In certain embodiments, the ATP detection reagent is a chemiluminescent system, including without limitation a luciferase/luciferin system. In certain embodiments, the luciferase is a surface-bound enzyme. The ATP produced by the polymerase reaction is consumed in the luciferin-luciferase reaction, resulting in the production of inorganic pyrophosphate and light. Thus, the amount of light produced is directly proportional to the amount of ATP released by the polymerase, which in turn is directly proportional to the number of ARNs incorporated into the nascent polynucleotide. In certain embodiments, the light generated by the luciferin-luciferase reaction is detected. Such detection methods are well-known and commonly employed in the art.

### METHODS

The present invention provides methods for the detection or quantification of a RNA virus sequences, comprising the steps of: contacting a sample suspected of containing the target virus sequence in a reaction mixture as described above; and measuring the change in signal from the ATP detection reagent(s), where the level of change is proportional to the amount of target sequence present in the sample. In some such embodiments the RNA virus is SARS-CoV2, where the primer sequences optionally include one or more of the primer sequences set forth in Table 1.

In such assays, a change in signal that results from the presence of released ATP, e.g. a fluorescent signal, light, etc. is generated by the DNA polymerization from the presence of the target polynucleotide in the sample. The signal is monitored and quantified with detectors, such as fluorescence spectrophotometers, microplate readers, UV lamps, PCR, commercial systems that allow the monitoring of fluorescence in real time reactions, or, in some instances, by the human eye. Where the detectable signal is light, e.g. from a luciferase based system, a wide range of lumimometer devices are commercially available for tubes, plates, multimodal plates, etc.

### KITS

Also provided are kits for practicing the subject methods. The kits according to the present invention may comprise at least a combination of ARN and dNTP reagents in concentrations and ratios suitable for use in the methods described herein. For any given base, the stock solution will contain a native dNTP or an ARN, but not both carrying the same nucleobase. A kit may further include reverse transcriptase. A kit may further include reagents for detecting ATP, including, but not limited to one or both of: (a) an ATP-responsive fluorescent dye; (b) a luciferase and luciferin. A kit may further include additional reagents employed in the methods of the invention, e.g., buffers, nuclease inhibitors, etc. In certain embodiments, the kits will further include instructions for practicing the subject methods or means for obtaining the same (e.g., a website URL directing the user to a webpage which provides the instructions), where these instructions may be printed on a substrate, where substrate may be one or more of: a package insert, the packaging, reagent containers and the like. In the subject kits, the one or more components are present in the same or different containers, as may be convenient or desirable. A kit may include a primer to initiate polymerase synthesis on a specific RNA virus target, e.g. SARS-CoV2, etc. Multiple primers may be included. The various reagent components of the kits may be present in separate containers, or may all be precombined into a reagent mixture for combination with samples.

### EXPERIMENTAL

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the scope of the invention.

### Methods

Preparation of RNA fragments with viral genomic sequences. Plasm ids (IDT) containing DNA sequences excerpted from the N regions of SARS-CoV-2, SARS-CoV, or MERS-CoV genomes were procured. RNA fragments (approximately 1.2 kb) of these viral genomes were produced by performing PCR followed by in vitro T7 RNA transcription with each respective plasmid.

Detection of SARS-CoV-2 RNA fragments. In order to specifically detect SARS-CoV-2 RNA sequences, ATP-releasing nucleotides (ARNs) were employed in conjunction with specific primers (IDT) and a reverse transcriptase enzyme (SuperScript IV reverse transcriptase) in a method referred to as polymerase-amplified release of ATP (POLARA). Greatest specificity for SARS-CoV-2 RNA sequences was achieved by mixing RNA (50 nM) and primers (1 μM) in a 0.2 mL tube, heating at 85° C. for 5 minutes, and immediately cooling on ice. After the addition of reaction buffer, nucleotides (20 μM each), and reverse transcriptase (0.5 μL), 10 μL reaction mixtures were incubated at 55° C. for 10 minutes. Tubes were then immediately cooled to ambient temperature by placing tubes in an aluminum block pre-equilibrated to room temperature. Each 10 μL reaction mixture was then pipetted into a 384-well plate (Greiner/white) and 10 μL Kinase-Glo reagent (Promega) was added to each well and mixed at room temperature. The 384-well plate was loaded into a microplate reader luminometer and luminescence signals were measured after 10 minutes.

### Results

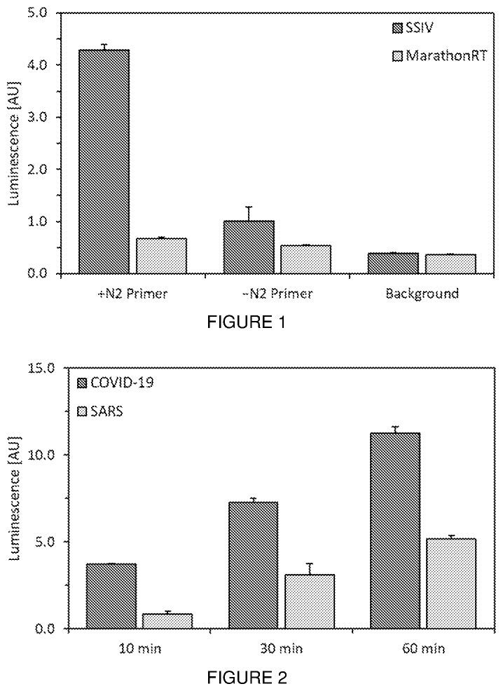

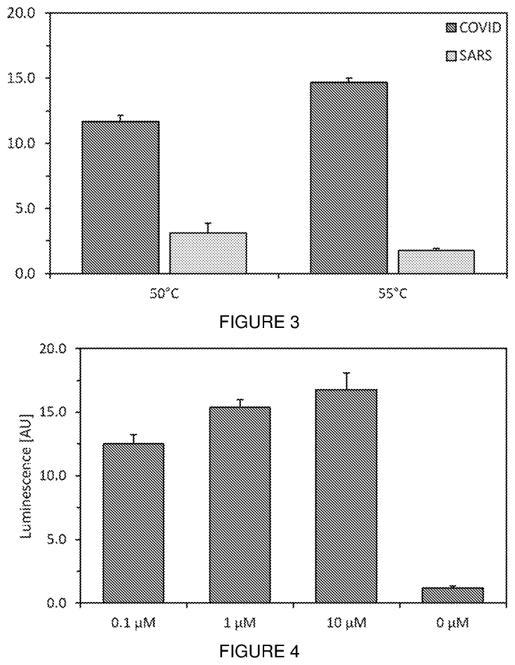

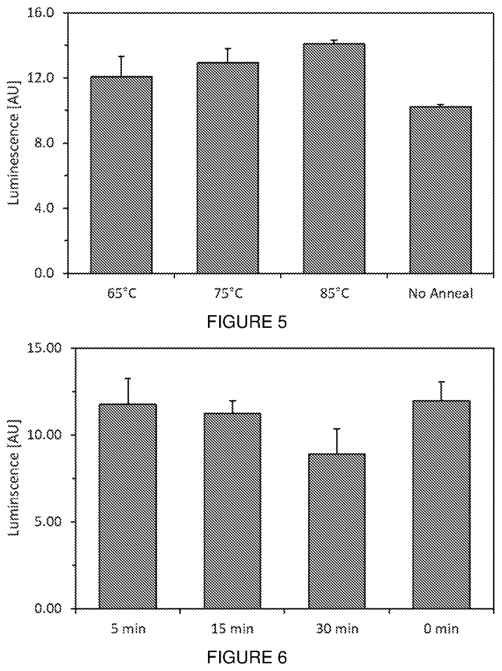

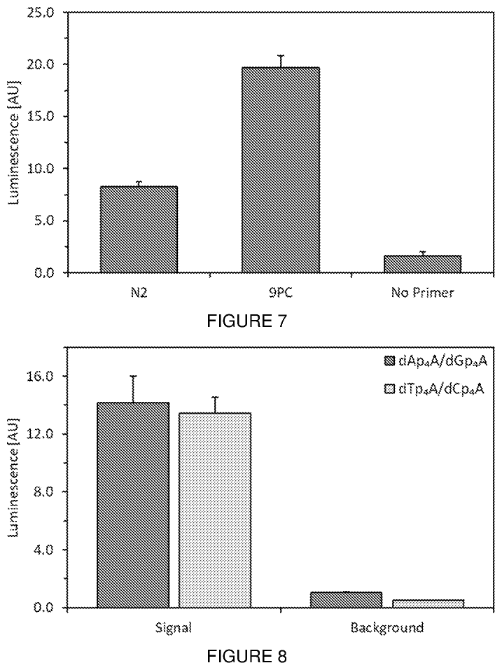

Optimization. In order to achieve both maximum specificity and sensitivity for SARS-CoV-2 RNA sequences several conditions had to be optimized. First, SuperScript IV was identified as a highly processive reverse transcriptase (RT) with the ability to efficiently accept ARNs as substrates (FIG. 1). A reaction time of 10 minutes was selected for extension as longer incubations resulted in a reduced selectivity (FIG. 2). Two extension temperatures (50° C. and 55° C.) suitable for SuperScript IV extension were tested; 55° C. provided superior selectivity and sensitivity (FIG. 3). Subsequently, it was found that heating and flash-cooling of RNA and primer(s) had a substantial positive effect on specificity. Using this annealing step, a primer concentration of 10 μM provided the highest sensitivity of the concentrations tested (FIG. 4). However, increasing primer concentration also reduces selectivity; in order to balance these contrasting effects, a primer concentration of 1 μM was used in subsequent experiments. Heating to 85° C. provided the highest sensitivity (greatest signal) of the temperatures tested, while increasing incubation time beyond 5 minutes resulted in a decrease in signal (FIG. 5). Thus, a pre-incubation step of 85° C. for 5 minutes was used in detection experiments, followed by immediate cooling on ice (FIG. 6). Adding a combination of 9 primers specific to SARS-CoV-2 also increased sensitivity (FIG. 7). The sequences of these 9 primers are listed in the primer sequences section (VSPs 1-7, N2, N3).

ARNs were investigated as a source of background signal. There are two possible sources of background resulting from ARNs: (1) residual ATP contamination from the insufficient purification, and (2) the potential for these nucleotides to be accepted as substrates by luciferase. While ATP contamination can be removed, the kinetics of a nucleotide to react with luciferase is intrinsic to the molecule and thus much more difficult to manipulate. Apyrase was used to deplete nucleotide stocks of ATP in order to compare their reactivity with luciferase. Purine derivatives (dAp₄A and dGp₄A) showed higher activity with luciferase than their pyrimidine counterparts (dCp₄A and dTp₄A). However, dCp₄A had high levels of ATP contamination. Using ATP-depleted dCp₄A along with dTp₄A, dATP, and dGTP resulted in improved signal-to-noise ratios compared to using a mixture of dAp₄A, dGp₄A, dCTP, and dTTP, which was previously identified as the optimal combination (FIG. 8).

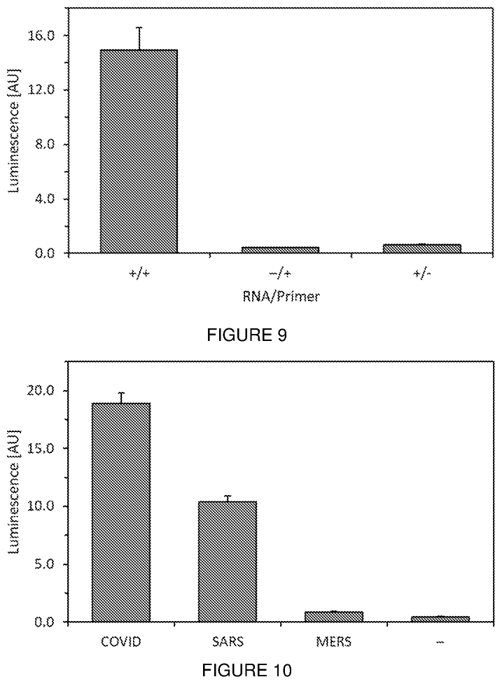

Detecting SARS-CoV-2 RNA Sequences in Total RNA. Using the developed method, 1.2 kb RNAs containing an excerpt from the N region of the SARS-CoV-2 genome could be detected at a concentration of 50 nM in the presence of 1 μg of total RNA isolated from human lung tissue with virtually no background signal (FIG. 9). This showed that background signal resulting from non-specific primer extension events is not a concern. Additionally, the SARS-CoV-2 RNA sequence specifically detected, with 1.8-fold lower signal SARS-CoV sequences and 22-fold for MERS-CoV sequences (FIG. 10). The specificity of this method may be further improved by optimizing the combination of primer sequences.

Primer Sequences Table 1

| Primer Name | Sequence |
|---|---|
| 2019-nCoV | (SEQ ID NO: 1) CCAATGCGCGACATTCCG |
| VSP-1 | |
| 2019-nCoV | (SEQ ID NO: 2) ACTTCCATGCCAATGCGC |
| VSP-2 | |
| 2019-nCoV | (SEQ ID NO: 3) CTCATGGATTGTTGCAAT |
| VSP-3 | |
| 2019-nCoV | (SEQ ID NO: 4) CTGCGGTAAGGCTTGAGT |
| VSP-4 | |
| 2019-nCoV | (SEQ ID NO: 5) AATTTGATGGCACCTGTG |
| VSP-5 | |
| 2019-nCoV | (SEQ ID NO: 6) TGAGTTGAGTCAGCACTG |
| VSP-6 | |
| 2019-nCoV | (SEQ ID NO: 7) TTGTGTTACATTGTATGC |
| VSP-7 | |
| 2019-nCoV | (SEQ ID NO: 8) GCGCGACATTCCGAAGAA |
| N2 | |
| 2019-nCoV | (SEQ ID NO: 9) TGTAGCACGATTGCAGCATTG |
| N3 | |

1. A method for detecting the presence of a virus RNA sequence in a sample comprising nucleic acids, the method comprising:
contacting the nucleic acid with a reaction mixture comprising:
at least one ATP-releasing nucleotide (ARN) having a structure 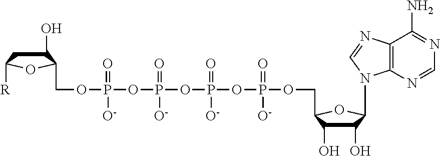
wherein R is where R is any purine or pyrimidine, or an analog thereof that retains an ability to base pair with a complementary nucleotide; and optionally dNTPs, wherein the combination of dNTPs and ARN is sufficient to provide a substrate for all bases present in the sequence of interest;
a primer complementary to the target virus RNA; and
a reverse transcriptase that incorporates ARNs; and
detecting the presence of ATP released during extension of the target by the reverse transcriptase.

2. The method of claim 1, wherein the virus is a coronavirus.

3. The method of claim 2, wherein the coronavirus is SARS-CoV2.

4. The method of claim 1, wherein the reaction mixture comprises a plurality of primers complementary to the virus RNA.

5. The method of claim 4, wherein the primer or primers are selected from the primers of Table 1, SEQ ID NO:1-SEQ ID NO:9.

6. The method of claim 5, wherein each of the primers present in Table 1 are present in the reaction mixture.

7. The method of claim 1, wherein the extension reaction proceeds for about 10 minutes.

8. The method of claim 1, wherein the reaction mixture comprises ATP-depleted dCp₄ A.

9. The method of claim 8, wherein the reaction mixture comprises as the sole nucleotides: ATP-depleted dCp₄A, dTp₄A, dATP, and dGTP.

10. The method of claim 1, wherein the reverse transcriptase is an ultra-processive reverse transcriptase.

11. The method of claim 10, wherein the reverse transcriptase is an engineered MMLV mutant RT.

12. The method of claim 1, wherein the extension reaction temperature is about 55° C.

13. The method of claim 1, wherein detecting ATP comprises the step of contacting the reaction mixture with luciferin and an ATP-dependent luciferase enzyme to produce light.

14. A reaction mixture for use in a method of claim 1.
